(19) United States
(12) Patent Application Publication
(10) Pub. No.: US 2025/0099739 A1
(43) Pub. Date: Mar. 27, 2025

(54) METHOD AND APPARATUS FOR DELIVERING AN OXYGENATED FLUID

(71) Applicant: ABIOMED, Inc., Danvers, MA (US)

(72) Inventors: Sonya Bhavsar, Danvers, MA (US); Alexandra Fearn, Danvers, MA (US); Andrea Merin, Danvers, MA (US)

(73) Assignee: ABIOMED, Inc., Danvers, MA (US)

(21) Appl. No.: 18/890,928

(22) Filed: Sep. 20, 2024

Related U.S. Application Data
(60) Provisional application No. 63/539,719, filed on Sep. 21, 2023.

(51) Int. Cl. A61M 60/38; A61M 60/13; A61M 60/17; A61M 60/216; A61M 60/829

(52) U.S. Cl. CPC A61M 60/38; A61M 60/13; A61M 60/17; A61M 60/216; A61M 60/829; A61M 2205/3633

(57) ABSTRACT

A blood pump that delivers an oxygenated-fluid assists with a patient's cardiovascular function by increasing the oxygenation of the blood. Disclosed is a system and method for delivering an oxygenated fluid. The system includes a blood pump, a catheter operably coupled to the blood pump, and a purge fluid line including a first lumen configured to deliver a purge fluid to a motor section of the blood pump, at least a portion of the purge fluid line extending along a catheter, wherein the purge fluid is an oxygenated purge fluid.

### CROSS-REFERENCE TO RELATED APPLICATIONS

The present application claims priority to U.S. Provisional Patent Application No. 63/539,719, filed Sep. 21, 2024, the contents of which are incorporated by reference herein in its entirety.

### TECHNICAL FIELD

Embodiments disclosed herein relate to medical devices, such as intravascular blood pumps, and devices and methods for delivering an oxygenated fluid (e.g., an oxygenated purge fluid) to a patient.

### BACKGROUND

Blood pump assemblies, such as intracardiac or intravascular blood pumps may be introduced in the heart to deliver blood from the heart into an artery. Such mechanical circulatory support devices are often introduced to support the function of the heart after a patient suffers a cardiac episode. One such class of devices is the set of devices known as the IMPELLA® heart pump. Some blood pump assemblies may be introduced percutaneously through the vascular system during a cardiac procedure. Specifically, blood pump assemblies can be inserted via a catheterization procedure through the femoral artery or through the axillary/subclavian artery, into the ascending aorta, across the valve and into the left ventricle. The inserted blood pump assembly may be configured to pull blood from the left ventricle of the heart through a cannula and expel the blood into the aorta. A blood pump assembly may also be configured to pull blood from the inferior vena cava and to expel blood into the pulmonary artery. Some mechanical circulatory support devices are powered by an on-board motor, while others are powered by an external motor and a drive cable.

### BRIEF SUMMARY

In various aspects, a purge fluid system may be provided. The purge fluid delivery system may include a blood pump. The purge fluid system may include a catheter. The catheter may be operably coupled to the blood pump. The purge fluid system may include a purge fluid line. The purge fluid line may include a first lumen. The first lumen may be configured to deliver a purge fluid to a motor section of the blood pump. At least a portion of the purge fluid line may extend along the catheter. The purge fluid may be an oxygenated purge fluid.

The purge fluid line of the purge fluid delivery system may include a distal end and a proximal end opposite to the distal end. The distal end of the purge fluid line may be operably coupled to a proximal end of the motor section.

The purge fluid line of the purge fluid delivery system may include a second lumen. The second lumen may be fluidly coupled to the first lumen and configured to deliver the purge fluid.

The purge fluid line of the purge fluid delivery system may be configured to be insulated along a length of the catheter to maintain a temperature of the purge fluid.

The purge fluid delivery system may include a controller. The controller may be configured to control the delivery of the purge fluid.

The first lumen of the purge fluid delivery system may extend into the motor section.

In various aspects, a system may include a blood pump. The system may include a delivery system. The delivery system may be configured to deliver an oxygenated fluid to at least one of the blood pump and a ventricle of the patient. The delivery system may include a fluid line having a first lumen.

The first lumen of the system may be configured to be insulated to maintain a temperature of the oxygenated fluid along a length of the first lumen. The fluid line of the system may include a purge fluid line. The purge fluid line may be operable coupled to a motor section of the blood pump. The oxygenated fluid may be configured to pass through the motor section of the blood pump. The oxygenated fluid of the system may be configured to exit the motor section into a ventricle of a patient.

The fluid line of the system may include an oxygenation line. The oxygenation line may be configured to extend along at least a portion of a catheter attached to the blood pump. The oxygenation line of the system is separate from the catheter of the blood pump. The blood pump of the system may include a purge fluid line extending through the catheter. The blood pump system may be configured to deliver a purge fluid to a motor of the blood pump.

The system may include an oxygenation system.

The system may include a controller. The controller may be configured to control the delivery of the oxygenated fluid. The controller of the system may control the blood pump. The controller may control the oxygenation system. The purge fluid line of the system may be configured to deliver an oxygenated purge fluid to a motor of the blood pump.

The oxygenated fluid passing through the oxygenation line of the system may include more dissolved oxygen than the oxygenated purge fluid.

In various aspects, a method for operating a blood pump may be provided. The method may include inserting a blood pump into a patient. The method may include operating the blood pump to cause an impeller in the blood pump to rotate based on a rotation of a shaft in a motor section by a motor in the motor section to move blood. The method may include providing an oxygenated fluid to at least one of the motor section of the pump or a ventricle of the patient. The method may include oxygenating a fluid via an oxygenation system.

The step of providing an oxygenated fluid of the method may include providing an oxygenated fluid through a first lumen of a purge fluid line extending along at least a portion of a catheter operably coupled to the blood pump. The step of providing the oxygenated fluid of the method may include providing an oxygenated fluid through a first lumen of an oxygenation line extending through at least a portion of a catheter operably coupled to the blood pump. The blood pump may have a purge fluid lune extending through the catheter.

The step of proving an oxygenated fluid of the method may include providing an oxygenated fluid to the first lumen of the purge fluid line and the first lumen of the oxygenation line. The step of proving an oxygenated fluid of the method may include providing an oxygenated fluid through a first lumen of an oxygenation line separate from the catheter.

The oxygenated purge fluid may have a dissolved oxygen content between about 30 mg/L and about 50 mg/L. The oxygenated purge fluid may have a dissolved oxygen content between about 35 mg/L and about 45 mg/L. The purge fluid may have a dissolved oxygen content between about 37 mg/L and about 42 mg/L.

The oxygenated purge fluid may be delivered at a temperature of between about 6 degrees Celsius and about 20 degrees Celsius. The oxygenated purge fluid may be delivered at a temperature of between about 10 degrees Celsius and about 16 degrees Celsius. The oxygenated purge fluid may be delivered at a temperature of between about 12 degrees Celsius and about 14 degrees Celsius.

### BRIEF DESCRIPTION OF THE FIGURES

FIG. 1 shows a patient's heart with a blood pump inserted into a left ventricle;

FIG. 2 shows a patient's heart with a blood pump inserted into a right ventricle;

FIG. 3 shows an illustrative side view of a blood pump;

FIG. 4 shows an illustrative view of a mechanical circulatory support system;

FIG. 5A shows an illustrative view of a first embodiment a mechanical circulatory support system with a second container;

FIG. 5B shows an illustrative view of a second embodiment of a mechanical circulatory support system with a second container;

FIG. 6A shows an illustrative side view of a catheter having multiple lumens;

FIG. 6B shows a patient's heart with a blood pump and purge fluid line inserted into the left ventricle; and

FIG. 7 illustrates a method according to one embodiment.

### DETAILED DESCRIPTION

As it is known, percutaneous blood pumps are inserted into a patient's vasculature to assist with cardiovascular function. For example, patients receiving support from such pumps may experience decreased oxygenation of blood as a result of poor cardiac performance and/or other conditions. In some instances, patients may receive oxygen via inhalation in order to improve blood oxygenation. As it is also known, dissolved supersaturated oxygen delivered intravenously may be used as a treatment of several patient conditions, including but not limited to acute myocardial infarction and/or hypoxia.

In some instances, blood pumps may include purged blood pumps that employ a purge fluid to maintain the motor section free of obstructions that could impair performance, or, in the long term, cause seizure of the pump. The purge fluid may enter the pump through the motor and exits into the patient's vasculature. Accordingly, the inventors have recognized the benefit of delivering oxygen-infused fluid to a vasculature of a patient. For example, as disclosed herein, an oxygen-infused purge fluid may be used to increase oxygenation of the blood. Such oxygen-infused purge fluid also may improve motor efficiency and cooling in some embodiments.

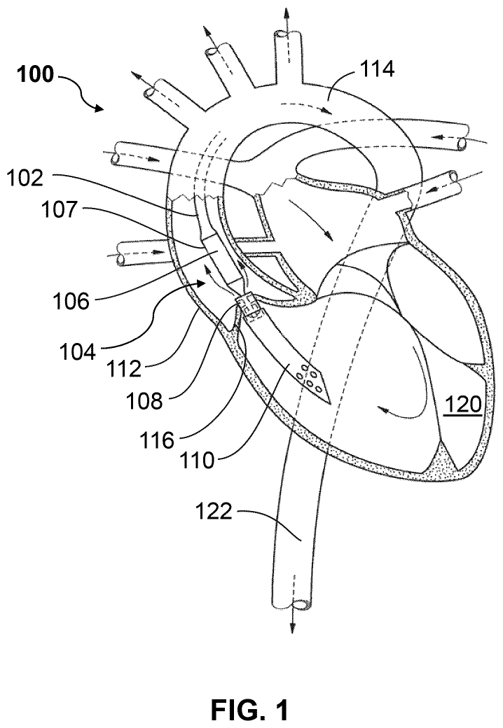

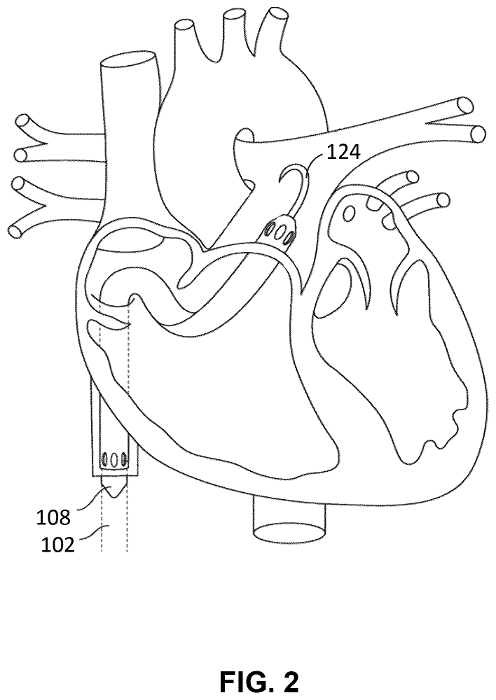

Turning to the figures, FIG. 1 shows a blood pump assembly (100) for use in a left ventricle. As shown in this view, the blood pump may include a catheter (102) and a pump housing (104) operably coupled to the catheter (102). The pump housing (104) may include a motor section (106) and a pump section (108) which may be disposed coaxially, one behind the other. In some embodiments, this may result in a rod-shaped construction form. The pump section (108) may have an extension in the form of a flexible suction hose (110), often referred to as cannula. An impeller may be provided in the pump section (108) to cause blood flow from a blood flow inlet to a blood flow outlet, and rotation of the impeller may be caused by an electric motor disposed in the motor section (106). The blood pump may be placed such that it lies primarily in the ascending aorta (112) leading to the aortic arch (114). The aortic valve (116) may come to lie, in the closed state, against the outer side of the pump section (108) or its suction hose (110) that lies substantially in the left ventricle (120). The blood pump (with the suction hose (110) in front) may be advanced into the represented position by advancing the catheter (102), optionally employing a guide wire. In so doing, the suction hose (110) may pass the aortic valve (116) retrograde, so the blood is sucked in through the suction hose (110) and pumped into the aorta (122). In some embodiments, the blood pump may be deployed in the right ventricle, as shown in FIG. 2.

As seen in blood pump assembly of FIG. 2, the pump housing (and pump section (108)) may be operably coupled to the catheter (102). As also shown in FIG. 2, in some embodiments, the pump may include an atraumatic extension (124), also referred to as a “pigtail”, which may be operably coupled to a distal end of the cannula (e.g., suction hose (110)) and may assist with stabilizing and/or positioning the blood pump assembly into the correct positioning the heart. The atraumatic extension (124) may be configurable from a straight to a partially curved (as shown) configuration. The atraumatic extension (124) also may be composed, at least in part, of a flexible material, and may have dual stiffness. As shown in FIG. 1, it should be appreciated that the pump assembly need not include an atraumatic extension (124). The catheter (102) may be operably coupled to an oxygenation system, as will be discussed herein.

As will be appreciated, the use of the blood pump is not restricted to the applications represented in FIGS. 1 and 2. For example, the pump can be inserted through the other peripheral vessels, such as the subclavian artery. Alternatively, reverse applications for the right ventricle may be envisioned.

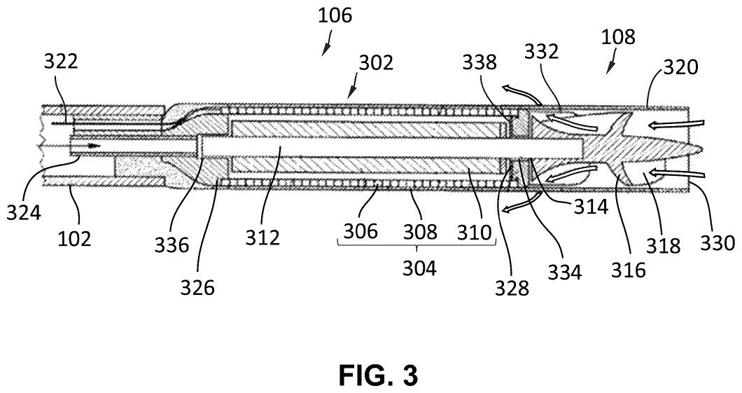

FIG. 3 shows an exemplary embodiment of the blood pump assembly (100). The motor section (106) of the blood pump assembly (100) may have an elongated housing (302) in which an electric motor (304) may be housed. A stator (306) of the electric motor (304) may have, in some embodiments, numerous circumferentially distributed windings as well as a magnetic return path (308) in the longitudinal direction. The magnetic return path (308) may form an outer cylindrical sleeve of the elongate housing (302). The stator (306) may surround a rotor (310) operably coupled to the motor shaft (312) and consisting of permanent magnets magnetized in the active direction. The motor shaft (312) may extend over the entire length of the motor housing (302) and protrude distally out of the latter through an opening (314). There, it carries an impeller (316) with pump vanes (318) projecting therefrom, which may rotate within a tubular pump housing (320) which may be firmly coupled to the motor housing (302).

The proximal end of the motor housing (302) may have the flexible catheter (102) attached (e.g., sealingly attached) thereto. Through the catheter (102), there may extend electrical cables (322) for power supply to and control of the electric motor (304). In addition, a purge fluid line (324) may extend through the catheter (102) and penetrate a proximal end wall (326) of the motor housing (302). Purge fluid may be fed through the purge fluid line (324) into the interior of the motor housing (302) and exit through the end wall (328) at the distal end of the motor housing (302). The purging pressure may be chosen such that it is higher than the blood pressure present, in order to thereby prevent blood from entering into the motor housing. For example, the purge pressure may be between 300 and 1400 mmHg depending on the case of application.

As described herein, the same purged seal can be combined with a pump that is driven by a flexible drive shaft and a remote motor.

Upon a rotation of the impeller (316), blood may be sucked in through the distal opening (330) of the pump housing (320) and conveyed backward within the pump housing (320) in the axial direction. Through radial outlet openings (332) in the pump housing (320), the blood may flow out of the pump section (108) and further along the motor housing (302). In some instances, this may allow the heat produced in the motor to be carried off. It also may be possible to operate the pump section with the reverse conveying direction, with blood being sucked in along the motor housing (302) and exiting from the distal opening (330) of the pump housing (320).

The motor shaft (312) may be mounted in radial bearings (334), (336) at the proximal end of the motor housing (302), on the one hand, and at the distal end of the motor housing (302), on the other hand. The radial bearings, in particular the radial bearing (334) in the opening (314) at the distal end of the motor housing, may be configured as sliding bearings. Furthermore, the motor shaft (312) may also be mounted axially in the motor housing (302), the axial bearing (338) likewise being configured as a sliding bearing. The axial sliding bearing (338) may serve for taking up axial forces of the motor shaft (312) which may act in the distal direction when the impeller (316) conveys blood from distal to proximal. Should the blood pump be used for conveying blood also or only in the reverse direction, a corresponding axial sliding bearing (338) may (also or only) be provided at the proximal end of the motor housing (302) in a corresponding manner. As will be appreciated, the blood pump may have other suitable arrangements in other embodiments. For example, in some embodiments, the pumps may include a ball-bearing rotor/stator system. Some systems may include ceramic bearings.

For purge blood pumps, the blood pump may be attached to a purge fluid source, with fluid passing into the motor housing through the purge fluid line. In some embodiments, such as that shown in FIG. 3, for example, the purge fluid may flow through the axial sliding bearing and further through the distal radial bearing. In the axial sliding bearing, the purge fluid may form a lubricating film in the bearing gap. The pressure at which the purge fluid flows through the motor housing has an adverse effect, however, on the width of the bearing gap. Specifically, higher purge fluid pressure requires a smaller bearing-gap width which results in a thinner lubricating film between the sliding surfaces. The thinner the lubricating film is, the greater the motor current that is required for driving the electric motor to overcome the frictional forces. This complicates the control of the blood pump, because the current conveying volume is normally established by stored characteristic curves solely on the basis of the motor current and the rotational speed (both known quantities). When the purge fluid pressure also affects the motor current, this factor also has to be taken into consideration. In view of the fact that the same blood-pump type can be operated for a great variety of applications with different purge fluid pressures between 300 and 1400 mmHg, it is important to avoid a dependence of motor current on purge fluid pressure. In some embodiments, purge flow rates may be in the range of about 2 mL/hour to about 30 mL/hour, such as between about 5 mL/hour and 20 mL/hour or between 2 ml/hour and 10 ml/hour, depending upon the pump type. In some embodiments, the purge pressure may be between about 1100 mmHg to about 300 mmHg.

The pump impeller does induce shear stress on the blood passing through the pump. Shear stress is induced predominantly in the gap between the impeller and the outer face of the ceramic bearing and between the impeller shaft and the inner race of the bearing [e.g., ceramic bearings, ball bearings, etc.]. Due to shear stresses to which the blood is subjected, blood proteins denature and polymerize as the blood passes through the pump. The deposition of denatured and agglomerated protein causes activation of the clotting cascade, which, in turn, causes the build-up of bio-deposits on the pump mechanisms (e.g., the impeller, the outflow cage, etc.). Small gaps between components (i.e., purge gaps) are particularly vulnerable to blockage by bio-deposits. The bio-deposit build-up will cause the motor current needed to operate the pump to increase. The increased motor current or bio-deposits can degrade pump performance or even cause a pump to stop.

To mitigate the adverse effects of shear on the blood that flows through the pump, the purge fluids used in purged blood pumps typically include an anticoagulant. A dextrose concentration determines the viscosity of the purge fluid and hence affects the purge flow rate. Purge fluids with lower dextrose concentrations are less viscous and flow more quickly with less pressure through the purge system. Purge fluids with higher dextrose concentrations (more viscous) result in a lower purge flow rate and require a greater purge pressure. A reduction in dextrose concentration from 20% to 5% results in an approximate 30% to 40% increase in purge flow rates.

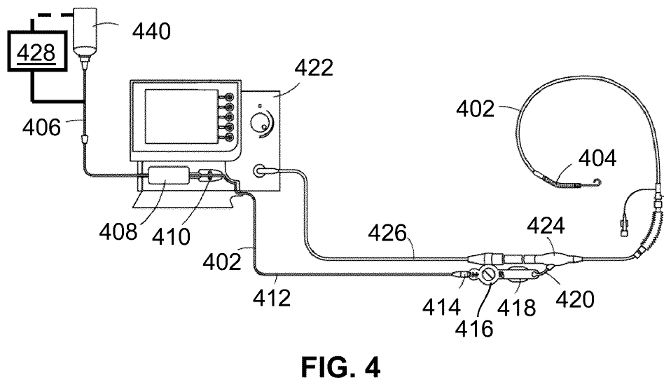

In one embodiment, the blood pump system may include a controller (422). In some embodiments, as shown in FIG. 4, the blood pump system may include a purge system subsystem (402), which may include a container (440), a supply line (406), a purge cassette (408), a purge disc (410), purge tubing (412), a check valve (414), a pressure reservoir (416), an infusion filter (418), sidearm (420), and repositioning unit (434). Container (440) may, for example, be a bag or a bottle. A purge fluid may be stored in the container (440). Supply line (406) may provide a fluidic connection between container (440) and purge cassette (408). Repositioning unit (434) may be used to reposition the blood pump. Additionally, as shown in FIG. 4, controller (422) may contain a display (432).

In some embodiments, purge cassette (408) may control how the purge fluid in container (440) is delivered to blood pump (404). For example, purge cassette (408) may include one or more valves (e.g., purge path diverters) for controlling a pressure and/or flow rate of the purge fluid. In addition to containing the components for delivering the purge fluid, purge cassette (408) may contain a rack and pinion which is attached to a piston. Purge disc (410) includes one or more pressure and/or flow sensors for measuring a pressure and/or flow rate of the purge fluid. As shown, controller (422) may interface with the purge cassette (408) and the purge disc (410). A sensor in controller (422) may measure the pressure so that the pressure can be displayed on the display (432). Controller (422) may include a stepper motor. A tic [or step] represents stepper motor positions in units of microsteps, which are also called pulses. In some embodiments, a purge pressure/purge flow curve algorithm can be deployed by the controller (422) along with the number of steps/minute of the stepper motor, and pressure measurement by purge disc (410) to calculate the corresponding purge flow rate. Purge tubing (412) may provide a fluidic connection between purge disc (410) and check valve (414). In some embodiments, a Y-connector and/or a yellow Luer container are also provided, which facilitate a continuous fluid path or purge fluid path. Y-connector is an adapter that connects purge tubing (412) to the blood pump (e.g., blood pump assembly (100)). A Luer connector (which may be, e.g., yellow) connects purge tubing (412) to a check valve (e.g., a yellow luer lock) on blood pump (404). Pressure reservoir (416) may provide additional filling volume and pressure during a purge fluid change. In some implementations, pressure reservoir (416) may include a flexible rubber diaphragm that provides the additional filling volume and pressure by means of an expansion chamber. An infusion filter (418) may help prevent bacterial contamination and air from entering catheter (102). Sidearm (420) provides a fluidic connection between infusion filter (418) and plug (424).

During operation, controller (422) may receive measurements from purge disc (410) and control the stepper motor's number of ticks to control the purge pressure. In some embodiments, during operation, purge cassette (408) is placed in controller (422) and connected with blood pump (404). As noted above, controller (422) may control and measure purge pressure and calculate purge flow rate via purge cassette (408) and/or purge disc (410). Controller (422) may also control the purge fluid supply. During operation, after exiting purging device through sidearm (420), the purge fluid is channeled through the purge lumens within catheter tube and plug. Sensor cables within catheter tube, connector cable, and plug provide an electrical connection between the motor within the motor housing (107) and controller (422). During operation, controller (422) may receive measurements from purge disc (410) through the sensor cables and controls the electrical power delivered to the motor within motor housing (107) through the motor cables. By controlling the power delivered to the motor within motor housing (107), controller (422) can control the speed of the motor within motor housing (107). In some embodiments, controller (422) includes safety features to prevent air from entering purge tubing (412). Controller (422) may include circuitry for monitoring the motor current for drops in current indicating air in the line. Controller (422) may include warning sounds, lights or indicators to alert an operator of disconnects or breaks in purge tubing (412) which may result in the introduction of air to the line. As described above, in this manner, the controller (422) may control the blood pump (404) and oxygenation system (428) by utilizing data measurements from at least one of the purge disc or purge cassette (408).

Any appropriate oxygenation system may be utilized here; oxygenation devices for various fluids are well known in the art, and may be, e.g., a membrane oxygenator.

As disclosed herein, in some embodiments, the pump system may be configured to deliver an oxygenated fluid to the patient. For example, in some embodiments, the oxygenated fluid may be delivered to the patient via the pump system. Accordingly, in such embodiments, an oxygenation system (428) may be operably coupled to the pump system. For example, in some embodiments, the oxygenation system may be fluidically coupled to the pump system. In some embodiments, the oxygenation system (428) may be operably (e.g., fluidly) coupled to container (440) storing a purge fluid and/or the supply line (406) of such container, as shown in FIG. 4. In some embodiments, the purge fluid in container (440) may be oxygenated (e.g., via oxygenation system (428)) before container (440) is coupled to the purge subsystem (402). In other embodiments, the purge fluid in container (440) may be oxygenated as the purge fluid travels from the container and to the pump system. As will be appreciated, the purge fluid may be oxygenated via any suitable system. For example, the oxygenation system (428) may include a gas/liquid diffusion head, which may be coupled to the purge fluid container (440) and/or to the controller (e.g., via the fluid supply line).

In an exemplary embodiment, the controller (422) display (432) may provide useful information to a user of the blood pump. Some of the information that may be displayed relate to the characteristics of the blood pump, such as, the serial number, the software version, etc., but also to the operation of the blood pump assembly, such as, the present blood pump speed (performance), blood pump flow measurements, purging device measurements, a status indicator, etc., Some of this information may be obtained from the purge disc (410) described above. Display (432) can also provide notifications to the user. For example, a notification may serve as an alert and include a statement describing the cause of the alert. In some embodiments, display (432) may be a touchscreen and a user may switch between screens by tapping button labels on display (432). In some embodiments, a user may use a separate input device, such as a mouse or a keyboard, to switch between screens.

As will be appreciated in view of the above, in one embodiment, the controller (422) may control the oxygenation system (428). In some embodiments, for example, the controller (422) may control the proportions of dissolved oxygen delivered to the purge fluid to oxygenate the purge fluid. The controller (422) also may control the frequency of delivery of dissolved oxygen. In an illustrative example, during operation, the controller (422) may receive a signal to oxygenate the purge fluid. Such signal may be in response to a prescribed time period a user (e.g., a clinician) chooses to deliver oxygenated fluid (e.g., oxygenated purge fluid) to the patient and/or on a prescribed concentration of oxygen to be delivered. In some embodiments, the user may select the desired delivery time and/or concentration via the control panel. In other embodiments, the signal may be responsive to a measured blood oxygen level of the patient. For example, in some embodiments, the pump system may be configured to measure the patient's blood oxygen levels, and when the measured level fall outside a prescribed range, the controller may instruct the oxygenation system to oxygenate the purge fluid for delivery to the patient. In still other embodiments, in response to other measurements received by the controller (e.g., cardiac performance, such as pressure measurements and/or flow rate of the purge fluid and/or the patient's blood), the controller may instruct the oxygenation system to oxygenate the purge fluid for delivery to the patient. As with the purge fluid, the controller may control the flow rate at which the oxygenated fluid is delivered to the patient (e.g., via the purge fluid line).

In embodiments in which the controller (422) adjusts the level of oxygenation of the purge fluid in response to measurements, the controller may receive such measurements through sensor cables. For example, sensor cables within catheter (102), connector cable (426), and/or plug (424) may provide an electrical connection between the motor within the motor housing (107) and controller (422). In some implementations, controller (422) may be operably coupled to an external power source (e.g., a battery or an electrical outlet of a power grid). In some implementations, controller (422) comprises an internal power source (e.g., a battery). When electric power is supplied by means of a battery, a patient may be afforded a greater degree of mobility.

In one embodiment, the oxygenated purge fluid may be cooled to a certain temperature before delivery to the patient. For example, the oxygenated purge fluid may be cooled to between 6 and 20 degrees Celsius, such as about 13 degrees Celsius. However, one skilled in the art may appreciate that the oxygenated purge fluid may be cooled to any suitable temperature. In some embodiments, the oxygenated purge fluid may be cooled to increase blood oxygenation. In some embodiments, the purge fluid delivery line may be insulated to maintain the desired temperature of the purge fluid along the length of the delivery line. The purge fluid delivery line may be insulated using any suitable means. For example, in some embodiments, the catheter (102) may have insulation around the circumference of the outside of the catheter. In other embodiments, only the purge fluid line (324) may be insulated. In still other embodiments, only the oxygenation fluid line (504) may be insulated, while the catheter (102) is not insulated. As will be appreciated, in some embodiments, may include insulating the supply line (406) from the fluid container (440), through the controller, and then from the controller to the pump system.

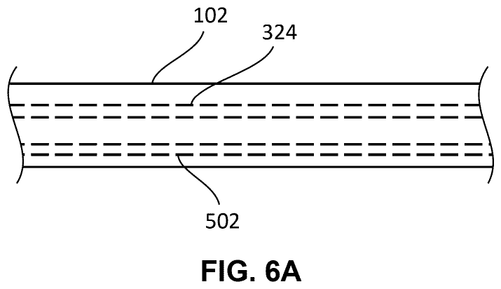

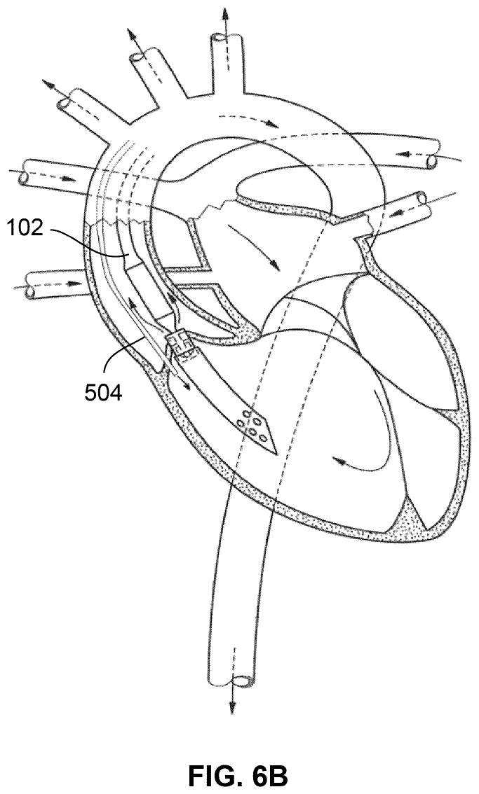

Although described as being delivered through the purge system (e.g., via purge fluid line (324) extending through the pump housing (302) in FIG. 3), it will be appreciated that the oxygenated fluid may be delivered via other suitable manner to the patient and via the pump system. For example, in some embodiments, dissolved oxygen may be delivered via another tube that is separate from the purge fluid delivery line. As seen in FIG. 6A, for example, catheter (102) may have a purge fluid line (324) that delivers purge fluid to the pump system (e.g., to prevent ingress of blood into the pump housing) and an oxygenation line, lumen (502), which may be a second oxygenation line, that extends through the pump system (e.g., through catheter (102)) to deliver the oxygenated fluid to the patient. In such embodiments, the oxygenated fluid may be configured to exit the blood pump system (e.g., such as via the outlets or other suitable portion of the pump system) and into the patients. As will be appreciated, although only a single oxygenation line is shown through the catheter in FIG. 6A, the blood pump system may have more than one oxygenation line in other embodiments.

In still another embodiment, as seen in FIG. 6B, dissolved oxygen may be delivered by an oxygenation line (504) that is separate from the catheter (102). For example, the oxygenation line may extend at least partially (sometimes fully) outside the pump system. As will be appreciated, although only one oxygenation line (504) is shown, some embodiments may include more than one line, or the oxygenation line (504) may include more than one lumen.

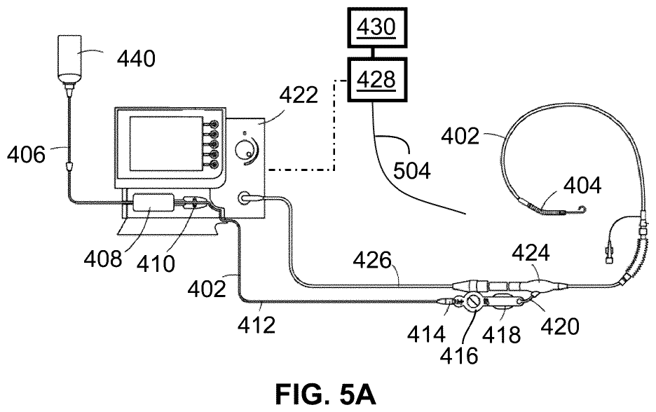

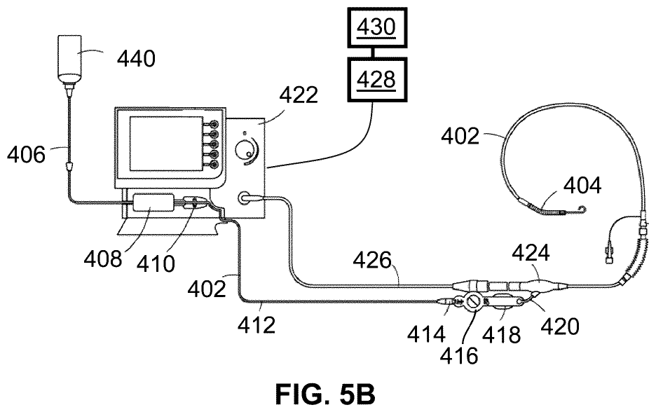

In embodiments where the oxygenation line is separate from the purge fluid line, the pump system may include a first container (440) having the purge fluid that is operably coupled to the purge fluid line, and a second container (430), as seen in FIGS. 5A and 5B, having an oxygenation fluid (e.g., a fluid that is pre-oxygenated or that is oxygenated after leaving the second container (430) en route to the patient) that is operably coupled to the oxygenation fluid line. In FIG. 5A, an embodiment is shown wherein a separate oxygenation fluid line (e.g., oxygenation fluid line (504)) is operably coupled to the oxygenation system (428), which is operably coupled to the second container (530). However, in addition to embodiments where the oxygenation fluid line is maintained separate from the catheter and/or blood pump, the second container may be operably coupled to the catheter and/or blood pump in various ways. One way is shown in FIG. 5B, where fluid from the second container (530) is directed towards the controller (422), before it is directed to the blood pump and/or the catheter (e.g., it may pass to the catheter and/or blood pump by passing from the controller through connector cable (426) or purge tubing (412).

In other embodiments, the same container (e.g., a purge fluid container (440)) may be used to supply both the purge fluid line and the oxygenation fluid line (e.g., fluidly coupled to both the purge fluid line and oxygenation fluid line). In such embodiments, the oxygenation system may be coupled to the container, such that the purge fluid is only oxygenated prior to entering the purge fluid line and not when the purge fluid enters the purge fluid line.

Also, in embodiments where the purge fluid line (324) is separate from the catheter (102) (as shown in FIG. 6B, for example), oxygenated fluid may be supplied to both the purge fluid line and the oxygenation line. As will be appreciated, in such embodiments, each of the purge fluid line and the oxygenation fluid line may have the same concentration of dissolved oxygenation in the fluid. In other embodiments, there may be more dissolved oxygen in the oxygenation fluid line than in the purge fluid line. As will be appreciated in view of the above, the controller may be configured to control the amount of oxygen in each of the lines. For example, the controller may instruct the oxygenation system to oxygenate fluid to a first concentration for the purge fluid line while instructing the oxygenation system to oxygenate fluid to a second concentration for the oxygenation fluid line. In such embodiment, the same oxygenation system may be used to oxygenate both the purge fluid line and the oxygenation fluid line. However, in other embodiments, multiple oxygenation systems may be operably coupled to the controller, each with a respective container, and each fluidly coupled to a single line (e.g., a respective purge fluid or oxygenated fluid line).

In the embodiments shown in FIGS. 6A and 6B, at least a portion of the length of the catheter (102) may be insulated, such that the temperature of the oxygenated fluid can be maintained along the length of the catheter. In some instances, the entire length of the catheter is insulated. In some embodiments a length of the catheter less than the entire length of the catheter is insulated. The catheter (102) may be insulated using any suitable method. For example, in some embodiments, the catheter (102) may have insulation around the circumference of the outside of the catheter. In other embodiments, only the purge fluid line (324) may be insulated. In still other embodiments, only the oxygenation fluid line (504) may be insulated, while the catheter (102) is not insulated.

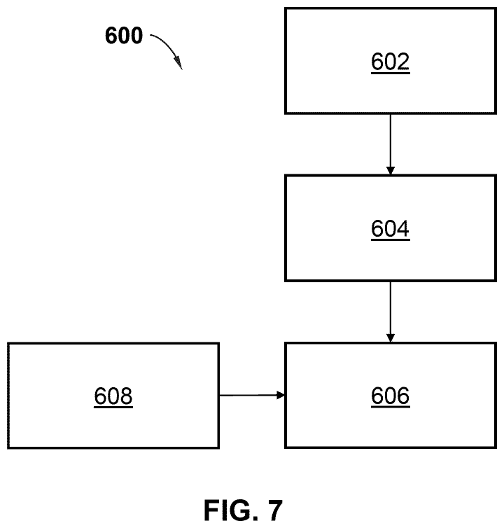

As shown in FIG. 7, the method (600) of providing oxygenated fluid to a patient is disclosed. As shown in this view, the method may include inserting (602) a blood pump into a patient. In some embodiments, the blood pump may include a pump housing with motor section.

The method also may include operating (604) the blood pump to cause an impeller in the blood pump to rotate based on a rotation of a shaft in the motor section by a motor in the motor section to move blood. The method also may include providing (606) an oxygenated fluid to the patient. In some embodiments, providing the oxygenated fluid may include providing the oxygenated fluid to at least one of the motor sections of the pump (e.g., which may thereafter exit the pump into the patient) or providing the oxygenated fluid directly to a ventricle of the patient. In some embodiments, this may include causing the oxygenated fluid to exit the motor section through the purge fluid line and enter a ventricle of a patient. In some embodiments, the step of providing an oxygenated fluid may include providing the oxygenated fluid through an oxygenation line extending through at least a portion of the catheter, the oxygenation line being separate from the purge fluid line. In still other embodiments, the step of providing an oxygenated fluid may include providing the oxygenated fluid through both the purge fluid line and the oxygenation fluid. In some embodiments, the method also may include preparing the oxygenated fluid (608), such as via an oxygenation system. In some embodiments, the step of preparing the oxygenated fluid may include oxygenating the fluid before attaching a container to a pump system. The step of preparing the oxygenated fluid also may include oxygenating the fluid during operation of the blood pump. In some embodiments, the method of preparing the oxygenated fluid may include controlling the oxygenation system via the controller, the controller also being used to control the blood pump through step (604) (e.g., operating the blood pump) and delivering the oxygenated fluid (e.g., in step (606)).

As will be appreciated in view of the above, the controller may control the oxygenation system and/or blood pump system to prepare and deliver the oxygenated fluid in response to parameters set by a user (e.g., a prescribed time and/or concentration). The controller also may control the oxygenation system and/or blood pump system to prepare and deliver the oxygenated fluid in response to parameters measured by the blood pump system and transmitted to the controller.

In some embodiments, the oxygenated purge fluid may be a dissolved oxygen content between about 30 mg/L and about 50 mg/L. In another embodiment, the oxygenated purge fluid may be a dissolved oxygen content between about 35 mg/L and about 45 mg/L. In another embodiment, the oxygenated purge fluid may be a dissolved oxygen content between about 37 mg/L and about 42 mg/L. In some embodiments, the oxygenated purge fluid is delivered at a temperature of between about 6 degrees Celsius and about 20 degrees Celsius. In another embodiment, the oxygenated purge fluid is delivered at a temperature of between 10 degrees Celsius and about 16 degrees Celsius. In still another embodiment, the purge fluid is delivered at a temperature of between 12 degrees Celsius and about 14 degrees Celsius.

Having thus described several aspects and embodiments of the technology set forth in the disclosure, it is to be appreciated that various alterations, modifications, and improvements will readily occur to those skilled in the art. Such alterations, modifications, and improvements are intended to be within the spirit and scope of the technology described herein. For example, those of ordinary skill in the art will readily envision a variety of other means and/or structures for performing the function and/or obtaining the results and/or one or more of the advantages described herein, and each of such variations and/or modifications is deemed to be within the scope of the embodiments described herein. Those skilled in the art will recognize, or be able to ascertain using no more than routine experimentation, many equivalents to the specific embodiments described herein. It is, therefore, to be understood that the foregoing embodiments are presented by way of example only and that, within the scope of the appended claims and equivalents thereto, inventive embodiments may be practiced otherwise than as specifically described. In addition, any combination of two or more features, systems, articles, materials, kits, and/or methods described herein, if such features, systems, articles, materials, kits, and/or methods are not mutually inconsistent, is included within the scope of the present disclosure.

The above-described embodiments can be implemented in any of numerous ways. One or more aspects and embodiments of the present disclosure involving the performance of processes or methods may utilize program instructions executable by a device (e.g., a computer, a processor, or other device) to perform, or control performance of, the processes or methods. In this respect, various inventive concepts may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other tangible computer storage medium) encoded with one or more programs that, when executed on one or more computers or other processors, perform methods that implement one or more of the various embodiments described above. The computer readable medium or media can be transportable, such that the program or programs stored thereon can be loaded onto one or more different computers or other processors to implement various ones of the aspects described above. In some embodiments, computer readable media may be non-transitory media.

The above-described embodiments of the present technology can be implemented in any of numerous ways. For example, the embodiments may be implemented using hardware, software or a combination thereof. When implemented in software, the software code can be executed on any suitable processor or collection of processors, whether provided in a single computer or distributed among multiple computers. It should be appreciated that any component or collection of components that perform the functions described above can be generically considered as a controller that controls the above-described function. A controller can be implemented in numerous ways, such as with dedicated hardware, or with general purpose hardware (e.g., one or more processor) that is programmed using microcode or software to perform the functions recited above, and may be implemented in a combination of ways when the controller corresponds to multiple components of a system.

Further, it should be appreciated that a computer may be embodied in any of a number of forms, such as a rack-mounted computer, a desktop computer, a laptop computer, or a tablet computer, as non-limiting examples. Additionally, a computer may be embedded in a device not generally regarded as a computer but with suitable processing capabilities, including a Personal Digital Assistant (PDA), a smartphone or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible formats.

Such computers may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Also, the phraseology and terminology used herein is for the purpose of description and should not be regarded as limiting. The use of “including”, “comprising”, or “having”, “containing”, “involving”, and variations thereof herein, is meant to encompass the items listed thereafter and equivalents thereof as well as additional items.

In the claims, as well as in the specification above, all transitional phrases such as “comprising,” “including”, “carrying”, “having”, “containing”, “involving”, “holding”, “composed of”, and the like are to be understood to be open-ended, i.e., to mean including but not limited to. Only the transitional phrases “consisting of” and “consisting essentially of” shall be closed or semi-closed transitional phrases, respectively.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

What is claimed is:

1. A purge fluid delivery system, comprising:
a blood pump;
a catheter operably coupled to the blood pump; and
a purge fluid line including a first lumen configured to deliver a purge fluid to a motor section of the blood pump, at least a portion of the purge fluid line extending along the catheter, wherein the purge fluid is an oxygenated purge fluid.

2. The purge fluid delivery system of claim 1, wherein the purge fluid line includes a distal end and a proximal end opposite to the distal end, the distal end of the purge fluid line operably coupled to a proximal end of the motor section.

3. The purge fluid delivery system of claim 1, wherein the purge fluid line includes a second lumen fluidly coupled to the first lumen and configured to deliver the purge fluid.

4. The purge fluid delivery system of claim 1, wherein the purge fluid line is configured to be insulated along a length of the catheter to maintain a temperature of the purge fluid.

5. The purge fluid delivery system of claim 1, further comprising a controller, the controller configured to control delivery of the purge fluid.

6. The purge fluid delivery system as in claim 3, wherein the first lumen extends into the motor section.

7. A system comprising:
a blood pump; and
a delivery system configured to deliver an oxygenated fluid to at least one of the blood pump and a ventricle of a patient, the delivery system including a fluid line having a first lumen.

8. The system of claim 7, wherein the first lumen is configured to be insulated to maintain a temperature of the oxygenated fluid along a length of the first lumen.

9. The system as in claim 7, wherein the fluid line includes a purge fluid line operably coupled to a motor section of the blood pump.

10. The system of claim 9, wherein the oxygenated fluid is configured to pass through the motor section of the blood pump.

11. The system of claim 10, wherein the oxygenated fluid is configured to exit the motor section into a ventricle of a patient.

12. The system as in claim 7, wherein the fluid line includes an oxygenation line configured to extend along at least a portion of a catheter attached to the blood pump.

13. The system as in claim 12, wherein the oxygenation line is separate from the catheter of the blood pump.

14. The system as in claim 12, wherein the blood pump includes a purge fluid line extending through the catheter and configured to deliver a purge fluid to a motor of the blood pump.

15. The system as in claim 7, further comprising an oxygenation system.

16. The system as in claim 7, further comprising a controller, the controller configured to control delivery of the oxygenated fluid.

17. The system of claim 16, wherein the controller controls the blood pump.

18. The system of claim 16, wherein the controller controls an oxygenation system.

19. The system of claim 14, wherein the purge fluid line is configured to deliver an oxygenated purge fluid to a motor of the blood pump.

20. (canceled)

21. A method for operating a blood pump, comprising:
inserting a blood pump into a patient;
operating the blood pump to cause an impeller in the blood pump to rotate based on a rotation of a shaft in a motor section by a motor in the motor section to move blood; and
providing an oxygenated fluid to at least one of the motor section of the blood pump or a ventricle of the patient.

22-32. (canceled)
